SEQUENCE LISTING (6 sequences)

SEQ ID NO: 1 - DNA, 8 bp, Artificial Sequence
  Inverse sequence of a docking section hybrid
gaaacttg

SEQ ID NO: 2 - DNA, 8 bp, Artificial Sequence
  Inverse sequence of a docking section hybrid
gttcaaag

SEQ ID NO: 3 - DNA, 10 bp, Artificial Sequence
  Human Papilloma Virus 6 detection probe
catatttatt

SEQ ID NO: 4 - DNA, 56 bp, Human papillomavirus type 6
ttggtgtatgtggaagatgtagttacggatgtacataatgtcatgttggtactgcg

SEQ ID NO: 5 - DNA, 38 bp, Artificial Sequence
  Human Papilloma Virus 6 detection probe
ttggtgtaagtggaagatgtagatacgtcaattttttt

SEQ ID NO: 6 - DNA, 27 bp, Human papillomavirus type 6
cgtaactacatcttccacatacaccaa

(19) United States
(12) Patent Application Publication
(10) Pub. No.: US 2010/0041030 A1
(43) Pub. Date: Feb. 18, 2010

(54) DISPLACEMENT ASSAY FOR DETECTING NUCLEIC ACID OLIGOMER HYBRIDIZATION EVENTS

(76) Inventor: Gerhard HARTWICH, Munchen (DE)

Correspondence Address: Pearl Cohen Zedek Latzer, LLP, 1500 Broadway, 12th Floor, New York, NY, 10036, US

(21) Appl. No.: 12/212,367

(22) Filed: Sep. 17, 2008

(30) Foreign Application Priority Data
Sep. 18, 2007 (DE) 10 2007 044 664.2

(51) Int. Cl. C12Q 1/68

(52) U.S. Cl. 435/6

(57) ABSTRACT

Described is a method for detecting nucleic acid oligomer hybridization events that comprises the steps providing a modified surface, the modification consisting in attaching at least one type of probe nucleic acid oligomer, providing at least one type of signal nucleic acid oligomer, the signal nucleic acid oligomers being modified with at least one detection label and the signal nucleic acid oligomers having a section that is complementary or largely complementary to the probe nucleic acid oligomers, providing a sample having target nucleic acid oligomers, bringing a defined quantity of the signal nucleic acid oligomers into contact with the modified surface, bringing the sample and the target nucleic acid oligomers contained therein into contact with the modified surface, detecting the signal nucleic acid oligomers and comparing the values obtained when detecting the signal nucleic acid oligomers with reference values. According to the present invention, the signal nucleic acid oligomers have a larger number of bases than the probe nucleic acid oligomers and exhibit at least one docking section, the docking section exhibiting no structure that is complementary or largely complementary to any section of the probe nucleic acid oligomers, and the target nucleic acid oligomers having a section that is complementary or largely complementary to the docking section.

### CROSS-REFERENCE TO RELATED APPLICATIONS

This application claims benefit of German Patent Application Ser. No. DE 10 2007 044 664.2, entitled “DISPLACEMENT ASSAY FOR DETECTING NUCLEIC ACID OLIGOMER HYBRIDIZATION EVENTS” filed Sep. 18, 2007, the entirety which is incorporated by reference herein.

### FIELD OF THE INVENTION

The present invention relates to a method for detecting nucleic acid oligomer hybridization events.

### BACKGROUND OF THE INVENTION

From WO 03/018834 A2 is known a displacement assay for detecting nucleic acid oligomer hybridization events that comprises the steps providing a modified surface, the modification consisting in attaching at least one type of probe nucleic acid oligomer, providing signal nucleic acid oligomers, providing a sample having target nucleic acid oligomers, bringing a defined quantity of the signal nucleic acid oligomers into contact with the modified surface and bringing the sample and the target nucleic acid oligomers contained therein into contact with the modified surface, detecting the signal nucleic acid oligomers, and comparing the values obtained when detecting the signal nucleic acid oligomers with reference values.

The fact that the displacement by the target nucleic acid oligomers of the signal nucleic acid oligomers hybridized to the probe nucleic acid oligomers exhibits a relatively low reaction rate proves to be a disadvantage of this method. Since, in the great majority of applications, the detection of the target nucleic acid oligomers is intended to occur in as short a time as possible, the slowly proceeding displacement of the signal nucleic acid oligomers by the target nucleic acid oligomers constitutes a serious disadvantage of the method described in WO 03/018834 A2.

### DESCRIPTION OF THE INVENTION

It is thus the object of the present invention to provide a displacement assay for detecting nucleic acid oligomer hybridization events that makes it possible to detect the target nucleic acid oligomers in a shorter period compared with the background art.

This object is solved by the method according to independent claim 1. Further advantageous details, aspects and embodiments of the present invention are evident from the dependent claims, the description, the drawings and the examples.

The following abbreviations and terms will be used in the context of the present invention:
DNA deoxyribonucleic acid
RNA ribonucleic acid
A adenine
G guanine
C cytosine
T thymine
U uracil
base A, G, T, C or U
bp base pair
nucleic acid At least two covalently linked nucleotides or at least two covalently linked pyrimidine (e.g. cytosine, thymine or uracil) or purine bases (e.g. adenine or guanine). The term nucleic acid refers to any backbone of the covalently joined pyrimidine or purine bases, such as the sugar-phosphate backbone of DNA, cDNA or RNA, a peptide backbone of PNA, or analogous structures (e.g. a phosphoramide, thiophosphate or dithiophosphate backbone). An essential feature of a nucleic acid is that it can sequence-specifically bind naturally occurring cDNA or RNA.
nucleotide, nt a monomeric component of a nucleic acid oligomer
oligonucleotide, oligo equivalent to nucleic acid oligomer, in other words e.g. a DNA, PNA or RNA fragment of a base length that is not further specified
sequence a nucleotide sequence in a nucleic acid oligomer
complementary To form the Watson-Crick structure of double-stranded nucleic acid oligomers, the two single strands hybridize, the nucleotide sequence of one strand being complementary to the nucleotide sequence of the other strand such that the A (or C) base of one strand forms hydrogen bonds with the T (or G) base of the other strand (in RNA, T is replaced by uracil).
mismatch To form the Watson-Crick structure of double-stranded nucleic acid oligomers, the two single strands hybridize in such a way that the base A (or C) of one strand forms hydrogen bonds with the base T (or G) of the other strand (in RNA, T is replaced by uracil). Any other base pairing within the hybrid does not form hydrogen bonds, distorts the structure and is referred to as a “mismatch”.
perfect match a hybrid composed of two complementary nucleic acid oligomers in which no mismatch occurs
ss single strand
ds double strand
redox-active Refers to the property of a moiety of giving up electrons to a suitable oxidizing agent or taking up electrons from a suitable reducing agent under certain external conditions.
EDTA ethylenediaminetetraacetate (sodium salt)
SNHS, sulfo-NHS N-hydroxysulfosuccinimide
NHS N-hydroxysuccinimide
EDC (3-dimethylaminopropyl)-carbodiimide
HEPES N-[2-hydroxyethyl]piperazine-N′-[2-ethanesulfonic acid]
tris tris(hydroxymethyl)aminomethane
linker, spacer A molecular link between two molecules or between a surface atom, surface molecule or surface molecule group and another molecule. Linkers can usually be purchased in the form of alkyl, alkenyl, alkynyl, heteroalkyl, heteroalkenyl or heteroalkynyl chains, the chain being derivatized in two places with (identical or different) reactive groups. These groups form a covalent chemical bond in simple/known chemical reactions with the appropriate reaction partner. The reactive groups may also be photoactivatable, i.e. the reactive groups are activated only by light of a specific or any given wavelength. Also unspecific nt, i.e. nt that is not complementary to other bases, can be used as linkers/spacers, especially in attaching probe oligos to a surface.

The present invention provides a method for detecting nucleic acid oligomer hybridization events that comprises the steps providing a modified surface, the modification consisting in attaching at least one type of probe nucleic acid oligomer, providing at least one type of signal nucleic acid oligomer, the signal nucleic acid oligomers being modified with at least one detection label and the signal nucleic acid oligomers having a section that is complementary or largely complementary to the probe nucleic acid oligomers, providing a sample having target nucleic acid oligomers, bringing a defined quantity of the signal nucleic acid oligomers into contact with the modified surface, bringing the sample and the target nucleic acid oligomers contained therein into contact with the modified surface, detecting the signal nucleic acid oligomers and comparing the values obtained when detecting the signal nucleic acid oligomers with reference values. According to the present invention, the signal nucleic acid oligomers have a larger number of bases than the probe nucleic acid oligomers and exhibit at least one docking section, the docking section exhibiting no structure that is complementary or largely complementary to any section of the probe nucleic acid oligomers, and the target nucleic acid oligomers having a section that is complementary or largely complementary to the docking section.

In the context of the present invention, a “largely complementary structure” is understood to be sequence sections in which no more than 20% of the base pairs form mismatches. In the context of the present invention, a “largely complementary structure” preferably means sequence sections in which no more than 15% of the base pairs form mismatches. Particularly preferably, a “largely complementary structure” means sequence sections in which no more than 10% of the base pairs form mismatches and very particularly preferably, sequence sections in which no more than 5% of the base pairs form mismatches.

Due to the docking section according to the present invention, the rate with which the association of the target nucleic acid oligomers to the signal nucleic acid oligomers occurs can be increased many times over. In the displacement assays for detecting nucleic acid oligomer hybridization events known from the background art, either probe nucleic acid oligomers and signal nucleic acid oligomers are present as a hybridized double strand when the target nucleic acid oligomers are added, or probe nucleic acid oligomers and target nucleic acid oligomers are present as a hybridized double strand when the signal nucleic acid oligomers are added. Accordingly, prio to a binding of the added nucleic acid oligomer component, the bonds of the hybridized double strand must be broken.

In contrast to this, according to the present invention, the two nucleic acid oligomer components, probe nucleic acid oligomers and signal nucleic acid oligomers, have a different number of bases. The signal nucleic acid oligomers exhibit a larger number of bases and provide a docking section that is present in a non-hybridized state, since it exhibits no structure that is complementary or largely complementary to any section of the probe nucleic acid oligomers.

At the same time, however, the target nucleic acid oligomers now exhibit a section that is complementary or largely complementary to the docking section. When the target nucleic acid oligomers are added, they may bind directly to this docking section without prior displacement of a hybridized component. In the course of the subsequent hybridization with the signal nucleic acid oligomers, the hybridized nucleic acid oligomer component must indeed be displaced, as known from the background art. However, due to the hybridization that already occurred with the docking section, this displacement occurs at a significantly higher rate.

Of course, the signal nucleic acid oligomers may exhibit two or even more docking sections. It is to be noted that the docking region need not connect directly to the section (“displace” region) of the signal nucleic acid oligomers that is complementary or largely complementary to the probe nucleic acid oligomers. Between the two regions, one section having any base sequence may be present, which can also form, for example, a loop.

The target nucleic acid oligomers are present in the sample either as a single strand (ss) or as a double strand (ds). In a preferred embodiment, the target nucleic acid oligomers are present, at least in part, as a single strand. This can be achieved, for example, in that ds target nucleic acid oligomers are dehybridized (thermally or through other measures known to the person of skill in the art) or in that, in preparing the target nucleic acid oligomers, care is taken that the target nucleic acid oligomers are present in part as single strands. This is achieved, for example, through an asymmetric PCR.

As already explained, the two nucleic acid oligomer components, probe nucleic acid oligomers and signal nucleic acid oligomers, have a different number of bases, the docking section being provided by the signal nucleic acid oligomers, which exhibit the larger number of bases. The target nucleic acid oligomers hybridize to the signal nucleic acid oligomers and, in this way, displace the previously bound probe oligomers. The hybridized double strand composed of target nucleic acid oligomers and signal nucleic acid oligomers detaches from the surface, at which non-hybridized probe oligomers remain. In detecting the signal oligomers, if the sought target is present, a decrease is observed in the signal intensity over time.

According to preferred embodiments of the present invention, the number of bases of probe nucleic acid oligomers and signal nucleic acid oligomers differs by 6 to 80, particularly preferably by 9 to 60, and especially preferably by 10 to 40. On the one hand, a length difference in the specified number of bases provides a sufficiently long docking section and, on the other hand, does not unnecessarily extend the nucleic acid oligomers used.

Advantageously, after a defined quantity of the signal nucleic acid oligomers is brought into contact with the modified surface, but before the sample and the target nucleic acid oligomers contained therein are brought into contact with the modified surface, a first detection of the signal nucleic acid oligomers is carried out to determine reference values. In the last step of the method according to the present invention, the values obtained in the further (second) detection of the signal nucleic acid oligomers are then compared with the reference values obtained in the first detection of the signal nucleic acid oligomers.

The signal nucleic acid oligomers are preferably modified with multiple detection labels, which results in signals with higher intensity being obtained. A fluorophore is particularly preferably used as the detection label, especially a fluorescent dye, such as especially Texas Red, a rhodamine dye or fluorescein.

According to a further preferred embodiment of the present invention, a redoxactive substance is used as the detection label. Likewise, a conductive surface is preferably used as the modified surface, which especially facilitates the electrochemical detection of the hybridization events.

Preferably, the detection of the signal nucleic acid oligomers occurs through a surface-sensitive detection method, since in this case only the signal nucleic acid oligomers that are bound to the surface are detected. Particularly preferred in this context are spectroscopic, electrochemical and electrochemiluminescent methods. As a spectroscopic method, particularly a detection of the fluorescence, especially the total internal reflection fluorescence (TIRF) of the signal nucleic acid oligomers is preferred.

For electrochemical detection, preferably cyclic voltammetry, amperometry, chronocoulometry, impedance measurement or scanning electrochemical microscopy (SECM) are used.

The signal nucleic acid oligomers preferably exhibit 10 to 200 nucleic acids, especially 20 to 100 nucleic acids, particularly preferably 25 to 70 nucleic acids. With the aid of signal nucleic acid oligomers of this length, all desired targets can be unambiguously identified.

Very particularly preferably, signal nucleic acid oligomers and probe nucleic acid oligomers are used that exhibit at least one non-complementary base pair in the region(s) of their sequence that hybridize(s) with each other. Thus, in this case, signal nucleic acid oligomers and probe nucleic acid oligomers exhibit a largely complementary structure within the meaning of the present invention. According to this embodiment, the displacement of the signal nucleic acid oligomers or of the probe nucleic acid oligomers by the target nucleic acid oligomers is greatly simplified, which leads, in turn, to an increased reaction rate.

The detection of the signal nucleic acid oligomers is preferably repeated multiple times, which makes it possible to determine the decrease in intensity over time in the course of the displacement of the signal nucleic acid oligomers or of the probe nucleic acid oligomers by the target nucleic acid oligomers. Here, the first detection of the signal nucleic acid oligomers preferably occurs in a buffer solution before the sample is brought into contact with the modified surface. Through this measurement, a reference value is obtained against which the subsequent measurements can be normalized after the target nucleic acid oligomers are added.

The first detection of the signal nucleic acid oligomers after the sample and the target nucleic acid oligomers contained therein are brought into contact with the modified surface occurs, independently of the question of whether a detection was carried out in a buffer solution or not, immediately after the sample is brought into contact with the modified surface and, thereafter, is repeated multiple times over a period of at least 40 min., preferably at least 20 min., particularly preferably at least 10 min. and very particularly preferably at least 5 min. In the cited periods, when detecting the signal nucleic acid oligomers, a sufficiently large intensity change normally occurs to be able to make a precise statement regarding the presence of a certain target nucleic acid oligomer.

The displacement process can be accelerated by a temperature increase. Thus, during the multiple repeating of the detection of the signal nucleic acid oligomers, the temperature in the region of the modified surface is preferably changed, starting at room temperature. Particularly good results are achieved when, during the repeated detection of the signal nucleic acid oligomers, the temperature in the region of the modified surface is raised above room temperature at a rate of 1° C. to 10° C. per min., preferably at a rate of 2° C. per min.

In an alternative form of the detection, during the multiple repeating of the detection of the signal nucleic acid oligomers, the temperature in the region of the modified surface is raised, starting from a first detection of the signal nucleic acid oligomers at room temperature, to a temperature that is particularly suitable for the detection, and the detection repeated multiple times at this temperature over a certain period of 2 min. to 60 min. Here, a particularly suitable temperature is considered to be a temperature that is 5° C. to 40° C. below the melting temperature of the hybrid composed of probe and signal oligonucleotides, especially one that is 5° C. to 30° C. below that, and a temperature that is 5° C. to 20° C. below the melting temperature of the hybrid is particularly preferred.

All methods described in the context of the present invention can be carried out using DNA chips. In this case, the modified surface exhibits at least 2 spatially substantially separated regions, preferably at least 4 and especially at least 12 spatially substantially separated regions.

The term “spatially substantially separated regions” is understood to mean regions on the surface that are quite predominantly modified by attaching a certain type of probe nucleic acid oligomer. Only in areas in which two such spatially substantially separated regions adjoin can it happen that different types of probe nucleic acid oligomers commingle.

Very particularly preferably, the modified surface exhibits at least 32, especially at least 64, and very particularly preferably at least 96 spatially substantially separated regions.

According to a further particularly preferred embodiment, in each case, one type of probe nucleic acid oligomer each is bound to the surface of one of the spatially substantially separated regions of the surface, the different types of probe nucleic acid oligomers differing from each other in at least one base. This permits the parallel detection of a number of different types of target nucleic acid oligomers.

The methods according to the present invention are particularly preferably carried out using a modified surface, the modification consisting in attaching at least one type of probe nucleic acid oligomer, wherein at least one type of signal nucleic acid oligomer modified with at least one detection label being present hybridized to the probe nucleic acid oligomers. Since, in this case, the signal nucleic acid oligomers are present already hybridized to the probe nucleic acid oligomers, the subsequent addition of the signal nucleic acid oligomers may be omitted. For the user of the method, this further simplifies the process.

The present invention also comprises a modified surface, the modification consisting in attaching at least one type of probe nucleic acid oligomer, wherein at least one type of signal nucleic acid oligomer modified at least with one detection label being present hybridized to the probe nucleic acid oligomers, the signal nucleic acid oligomers having a larger number of bases than the probe nucleic acid oligomers and the signal nucleic acid oligomers exhibiting at least one docking section, the docking section exhibiting no structure that is complementary or largely complementary to a section of the probe nucleic acid oligomers.

The number of bases of probe nucleic acid oligomers and signal nucleic acid oligomers preferably differs by 6 to 80, particularly preferably by 9 to 60 and very particularly preferably by 10 to 40. Likewise preferred are embodiments in which the signal nucleic acid oligomers are modified with multiple detection labels.

Particularly preferred are modified surfaces in which the signal nucleic acid oligomers bound to the surface carry a fluorophore as the detection label, especially a fluorescent dye, such as especially Texas Red, a rhodamine dye or fluorescein. But likewise preferably, also a redox-active substance may be used as the detection label. Since, in this case, an electrochemical detection is carried out, the modified surface is preferably a conductive surface.

The signal nucleic acid oligomers preferably exhibit 10 to 200 nucleic acids, especially 20 to 100 nucleic acids, particularly preferably 25 to 70 nucleic acids.

Signal nucleic acid oligomers and probe nucleic acid oligomers preferably exhibit at least one non-complementary base pair in the region(s) of their sequence that hybridize(s) with each other.

The modified surface preferably has at least 2 spatially substantially separated regions, particularly preferably at least 4 and especially at least 12 such spatially substantially separated regions. Very particularly preferably, the modified surface exhibits at least 32, especially at least 64, very particularly preferably at least 96 spatially substantially separated regions.

According to a preferred embodiment, in each case, one type of probe nucleic acid oligomer each is bound to the surface of one of the spatially substantially separated regions of the surface, the different types of probe nucleic acid oligomers differing from each other in at least one base.

In principle, the same advantages are associated with the various preferred embodiments of the modified surface according to the present invention as with the corresponding preferred embodiments of the methods according to the present invention. In this context, reference is made to the above explanations in connection with the methods according to the present invention.

In addition, the present invention also relates to a kit for carrying out one of the above-described methods for detecting nucleic acid oligomer hybridization events. The kit comprises a modified surface, the modification consisting in attaching at least one type of probe nucleic acid oligomer, and an effective quantity of signal nucleic acid oligomers having the properties explained in greater detail above.

In addition, the present invention also relates to a kit for carrying out one of the above-described methods for detecting nucleic acid oligomer hybridization events. The kit comprises a modified surface, the modification consisting in attaching at least one type of probe nucleic acid oligomer, wherein at least one type of signal nucleic acid oligomer modified at least with one detection label being present hybridized to the probe nucleic acid oligomers, the signal nucleic acid oligomers having a larger number of bases than the probe nucleic acid oligomers and the signal nucleic acid oligomers exhibiting at least one docking section, the docking section exhibiting no structure that is complementary or largely complementary to a section of the probe nucleic acid oligomers.

According to a preferred embodiment, the reference values are already comprised by the kit, such that the signal nucleic acid oligomers must be detected by the end consumer only once. The values obtained from this detection then need only be compared with the already existing reference values.

### The Surface

The term “surface” refers to any support material that is suitable for binding derivatized or non-derivatized probe nucleic acid oligomers covalently or via other specific interactions, directly or following appropriate chemical modification. The solid support may consist of conductive or non-conductive material.

### (i) Conductive Surfaces

The term “conductive surface” is understood to mean any support having an electrically conductive surface of any thickness, especially surfaces composed of platinum, palladium, gold, cadmium, mercury, nickel, zinc, carbon, silver, copper, iron, lead, aluminum and manganese.

In addition, any doped or undoped semiconductor surfaces of any thickness may also be used. All semiconductors may be used in the form of pure substances or in the form of composites. Examples include, but are not limited to, carbon, silicon, germanium, ? tin, and Cu(I) and Ag(I) halides of any crystal structure. All binary compounds of any composition and any structure composed of the elements of groups 14 and 16, the elements of groups 13 and 15, and the elements of groups 15 and 16 are likewise suitable. In addition, ternary compounds of any composition and any structure composed of the elements of groups 11, 13 and 16 or the elements of groups 12, 13 and 16 may be used. The designations of the groups of the periodic table of the elements are based on the IUPAC recommendation of 1985.

### (ii) Non-Conductive Surfaces

In the non-conductive surfaces, glass, modified glass or silicon is preferred as the material. The modification may take place e.g. by silanization and, in all cases, results in functional groups that are suitable for binding appropriately functionalized probe nucleic acid oligomers in coupling reactions. This modification includes layered superstructures on the surface, using polymers, such as dextran polymers, that permit a variation of the layer thickness and surface condition. Further derivatization possibilities for ultimately attaching the probe nucleic acid oligomers consist, for example, in applying a thin (approximately 10-200 nm) metallization layer, especially a gold metallization layer, which may additionally be coated with (thiol-functionalized) polymers, especially dextrans. In addition, following silanization, the glass may also be functionalized with biotin (e.g. amino-functionalized glass surface following silanization and coupling of the carboxylic acid biotin via a biotin active ester such as biotin-N-succinimidyl ester) or, alternatively, coated with a dextran lysine or dextran-immobilized biotin. Thereafter, the biotinylated glass surfaces produced in this way are treated with avidin or streptavidin and may then be used for attaching biotinylated probe nucleic acid oligomers.

### Binding Nucleic Acid Oligomers to the Surface

Methods for immobilizing nucleic acid oligomers on a surface are known to the person of skill in the art. The probe nucleic acid oligomers may be, for example, covalently bound to the surface via hydroxyl, epoxide, amino or carboxy groups of the support material with thiol, hydroxy, amino or carboxyl groups that are naturally present on the probe nucleic acid oligomer or that have been affixed to the probe nucleic acid oligomer by derivatization. The probe nucleic acid oligomer may be bound to the surface atoms or molecules of a surface directly or via a linker/spacer. In addition, the probe nucleic acid oligomer may be anchored by the methods common in immunoassays, such as by using biotinylated probe nucleic acid oligomers for non-covalent immobilization to avidin or streptavidin-modified surfaces. The chemical modification of the probe nucleic acid oligomers with a surface anchor group may already be introduced in the course of the automated solid-phase synthesis, or in separate reaction steps. Here, the nucleic acid oligomer is also linked directly or via a linker/spacer with the surface atoms or surface molecules of a surface of the type described above. This binding may be carried out in various ways known to the person of skill in the art. In this context, reference is made to WO 00/42217 A1.

### Probe, Target and Signal Nucleic Acid Oligomers

The probe nucleic acid oligomers of the present invention consist of nucleotides in a certain nucleotide sequence and are present immobilized on a surface. Molecules that specifically interact with the probe nucleic acid oligomers or with the signal nucleic acid oligomers to form a double-strand hybrid are referred to as target nucleic acid oligomers. Target nucleic acid oligomers within the meaning of the present invention are thus nucleic acid oligomers that function as complex binding partners of the complementary probe nucleic acid oligomer or signal nucleic acid oligomer. The target nucleic acid oligomers whose presence is to be detected based on the present invention exhibit at least one sequence region whose sequence is complementary or at least largely complementary to a section of the probe nucleic acid oligomers or of the signal nucleic acid oligomers.

The target nucleic acid oligomers are present in the sample either as a single strand (ss) or as a double strand (ds). In a preferred embodiment, the target nucleic acid oligomers are present, at least in part, as a single strand. This can be achieved, for example, in that ds target nucleic acid oligomers are dehybridized (thermally or through other measures known to the person of skill in the art) or in that, in preparing the target nucleic acid oligomers, care is taken that the target nucleic acid oligomers are present in part as single strands. This is achieved, for example, through an asymmetric PCR.

In the context of the present invention, a compound composed of at least two covalently joined nucleotides or at least two covalently joined pyrimidine (e.g. cytosine, thymine or uracil) or purine bases (e.g. adenine or guanine), preferably a DNA, RNA or PNA fragment, is used as the nucleic acid oligomer, or an oligomer. The term nucleic acid refers to any backbone of the covalently joined pyrimidine or purine bases, such as the sugar-phosphate backbone of DNA, cDNA or RNA, a peptide backbone of PNA, or analogous backbone structures, such as a thiophosphate, a dithiophosphate or a phosphoramide backbone. An essential feature of a nucleic acid within the meaning of the present invention is the sequence-specific binding of naturally occurring DNA, or RNA or structures derived (transcribed or amplified) therefrom, such as cDNA or amplified cDNA or amplified RNA (aRNA).

### Detection Label/Marker (Marker Molecule)

Through derivatization, the signal nucleic acid oligomers are provided with one or more detectable labels. This label makes it possible to detect the complexation events between the signal nucleic acid oligomer and the surface-bound probe nucleic acid oligomers. The label can supply a detection signal directly or, as in the case of enzyme-catalyzed reactions, indirectly. Preferred detection labels (marker molecules) are fluorophores and redox-active substances.

In the fluorophores, commercially available fluorescent dyes such as Texas Red, rhodamine dyes, fluorescein, etc. may be used (cf. Molecular Probes Catalog). In the event of detection by electrochemical methods, redox molecules are used as labels.

Transition metal complexes, especially those of copper, iron, ruthenium, osmium or titanium, may be used as redox labels with ligands such as pyridine, 4,7-dimethylphenanthroline, 9,10-phenanthrene quinonediimine, porphyrins and substituted porphyrin derivatives. In addition, it is possible to use riboflavin, quinones such as pyrrolloquinoline quinone, ubiquinone, anthraquinone, naphthoquinone or menaquinone, or derivatives thereof, metallocenes and metallocene derivatives such as ferrocenes and ferrocene derivatives and cobaltocenes and cobaltocene derivatives, porphyrins, methylene blue, daunomycin, dopamine derivatives, hydroquinone derivatives (para- or ortho-dihydroxybenzene derivatives, para- or ortho-dihydroxyanthraquinone derivatives, para- or ortho-dihydroxynaphthoquinone derivatives) and similar compounds.

In the methods according to the present invention, also indirect labels may be used. The term “indirect label” is understood to mean those in which the actual detectable form of the label is created only through an enzyme-catalyzed reaction. The detectable form of the label can then be detected on the surface. Examples of such indirect labels are known to the person of skill in the art from the literature, citing here by way of example alkaline phosphatase (AP) in connection with the substrate p-aminophenyl phosphate. If AP is present bound to the signal nucleic acid oligomer as an indirect marker, then an electrochemical detection of the signal nucleic acid oligomer may occur in that p-aminophenyl phosphate is added at the time of detection. The electrochemically inactive p-aminophenyl phosphate serves as a substrate of the enzyme AP and is converted to p-aminophenol. P-aminophenol may now, after diffusion to a conductive surface, be electrochemically detected, since this form of the substrate (that is, after conversion at AP) is electrochemically active. Alternatively, AP may also be used for chromogenic detection (e.g. with 5-bromo-4-chloro-3-indoxyl phosphate in connection with nitroblue tetrazolium chloride).

### Surface-Sensitive Detection Methods

Surface-sensitive detection methods permit distinguishing between marker molecules associated to a surface and those dissolved in excess. Electrochemical, spectroscopic and electrochemiluminescent methods are suitable as the detection method. For the embodiments described here, surface-sensitive detection methods are not compulsory since, in the described embodiments, the concentration of the signal oligonucleotides in the volume phase is normally comparatively low: It is begun with (labeled) signal oligonucleotides bound on the surface, which are successively detached from the surface in the course of the detection and released into the volume phase. The concentration of the signal oligonucleotides in the volume phase is thus low (especially at the beginning of the detection) and normally leads to no detectable distortion of the measurement signal by contributions from the volume phase. However, the use of surface-sensitive detection methods can increase the precision of the measurement result.

### (i) Surface-Sensitive Electrochemical Detection

In electrochemical methods, in principle, the kinetics of the electrochemical processes may be used to distinguish between redox-active detection labels adsorbed to a surface and those dissolved in excess. Generally, surface-adsorbed detection labels are electrochemically converted (e.g. oxidized or reduced) more quickly than redox-active detection labels from the volume phase, since the latter must first diffuse to the (electrode) surface before electrochemical conversion. Electrochemical surface-sensitive methods include cyclic voltammetry, amperometry and chronocoulometry, to mention some examples.

The chronocoulometry method, for example, permits differentiation of surface-near redox-active components from (identical) redox-active components in the volume phase and is described, for example, in Steel, A. B., Herne, T. M. and Tarlov M. J.: Electrochemical Quantitation of DNA Immobilized on Gold, Analytical Chemistry, 1998, Vol. 70, 4670-4677 and the literature cited therein. The use of chronocoulometry in a displacement assay for detecting nucleic acid oligomer hybridization events is described in detail in WO 03/018834 A2, to which reference is hereby made in this context.

### (ii) Surface-Sensitive Fluorescence Detection

Total internal reflection fluorescence (TIRF, cf. Sutherland and Dahne, 1987, J. Immunol. Meth., 74, 253-265) may serve as an optical measurement method for detecting fluorescence-labeled signal nucleic acid oligomers. Here, fluorescent molecules that are located near the interface between a solid waveguide medium, typically glass, and a liquid medium, or that are immobilized on the waveguide medium surface facing the liquid, can be excited by the evanescent field protruding from the waveguide and emit detectable fluorescent light. Fluorescence-labeled complex formers that are displaced or dissolved in the excess are not captured by the evanescent field (or only to the extent that they are located in the range of the penetration depth of the evanescent field) and thus contribute (nearly) nothing to the measured signal. The penetration depth of the evanescent field is typically 100 to 200 nm, but it may be increased by a thin metallization layer (approximately 10 to 200 nm), especially a gold metallization layer, to several 100 nm. In a preferred embodiment of the fluorescence detection of the displaced, fluorophore-labeled signal nucleic acid oligomers, the layer thickness of the probe-modified support surface is adjusted to the penetration depth of the evanescent field, e.g.
by appropriately long probe nucleic acid oligomers,
by immobilizing the probe nucleic acid oligomers via appropriately long linkers between the surface and the probe oligonucleotide,
by coupling the carboxylic acid biotin (via a biotin active ester such as biotin-N-succinimidyl ester) to amino-derivatized surfaces and coupling avidin or streptavidin to the biotinylated surfaces produced in this way, with subsequent attachment of biotinylated probe nucleic acid oligomers or
by immobilizing an appropriately thick layer of functionalized polymer and attaching the probe nucleic acid oligomer to the polymer, for example (a) by applying a thin (approximately 10-200 nm) metallization layer, especially a gold metallization layer, which may be coated with a (thiol-functionalized) polymer, especially dextrans or polylysine, which, in turn, is used for attaching the probe nucleic acid oligomers, or (b) by applying a polymer layer composed of polylysine biotin, dextran-lysine biotin or dextran-immobilized biotin, and coupling avidin or streptavidin to the biotinylated surfaces produced in this way, with subsequent attachment of biotinylated probe nucleic acid oligomers.

### BRIEF DESCRIPTION OF THE DRAWINGS

The invention will be explained in greater detail below by reference to exemplary embodiments in association with the drawings. Shown are:

FIG. 1 diagrammed schematically, a chip for detecting nucleic acid oligomer hybridization events by means of displacement assay;

FIG. 2 a schematic diagram of the individual steps of the method according to the present invention;

FIG. 3A a target nucleic acid oligomer (sequence 5′ to 3′);

FIG. 3B variants of the modified surface (i), (ii), (iv) and (v) according to the present invention with an on-chip reference (iii);

FIG. 4A measurement results of a displacement assay for an HPV_{—}6 target: Path of a detection sequence for an HPV-6 test site (-o-) whose probe and signal nucleic acid oligomers correspond to the diagram in FIG. 3B (ii) (signal nucleic acid oligomer with docking site), compared with a reference test site [symbol] whose probe and signal nucleic acid oligomers correspond to the diagram in FIG. 3B (iii). No HPV-6 target is contained in the analyte. The electrochemically detected signal of the ferrocene-labeled signal nucleic acid oligomer is plotted during application of a temperature gradient of 2° C./min to the test site, relative to the signal achieved at 26° C.

FIG. 4B measurement results of a displacement assay for an HPV_{—}6 target: measurement as described for FIG. 4A, but with 10 nM of target in the analyte solution (all other parameters are identical)

FIG. 5 measurement results of a displacement assay for an HPV_{—}6 target: measurement as described for FIG. 4B. Instead of a temperature gradient, a temperature jump to 38° C. was carried out after the reference measurement at 26° C., and the further measuring points at this temperature obtained as a function of time. All values are relative to the signal achieved at 26° C.

### REFERENCE SIGNS

I: Array of test sites (top view)
II: Test site in cross section
III: Immobilization of probe nucleic acid oligomers on a test site (e.g. via thiol-functionalized probe nucleic acid oligomers that are bound to gold test sites, section of a test site)
IV: Immobilization of signal nucleic acid oligomer by hybridization to probe nucleic acid oligomers (here: signal nucleic acid oligomers have a longer sequence than the probe nucleic acid oligomers; the region that extends beyond the probe nucleic acid oligomers is referred to as the docking region for the target)
1: Test site for attaching probes
2: Test site delimitation (not suitable for attaching probes)
3: Probe nucleic acid oligomer
4: Signal nucleic acid oligomer
5: Marker (here ferrocene) that is covalently bound to the signal nucleic acid oligomer
6: Hybrid composed of signal nucleic acid oligomer and target nucleic acid oligomer
101: Sequence region “upstream” from 102, can be used for hybridization to the docking section of the signal nucleic acid oligomer
102: Sequence that preferably serves to discriminate from other targets
103: Sequence region “downstream” from 102, can be used for hybridization to the docking section of the signal nucleic acid oligomer
201: Anchor on the surface (e.g. multiple thiol functions)
202: Linkers/spacers (e.g. multiple unspecific bases) do not hybridize with sequence sections of the signal and also not with those of the target nucleic acid oligomer
203: Marker/label (e.g. ferrocene, covalently bound to 205)
204: Probe nucleic acid oligomer
205: Signal nucleic acid oligomer

### Manner of Executing the Invention

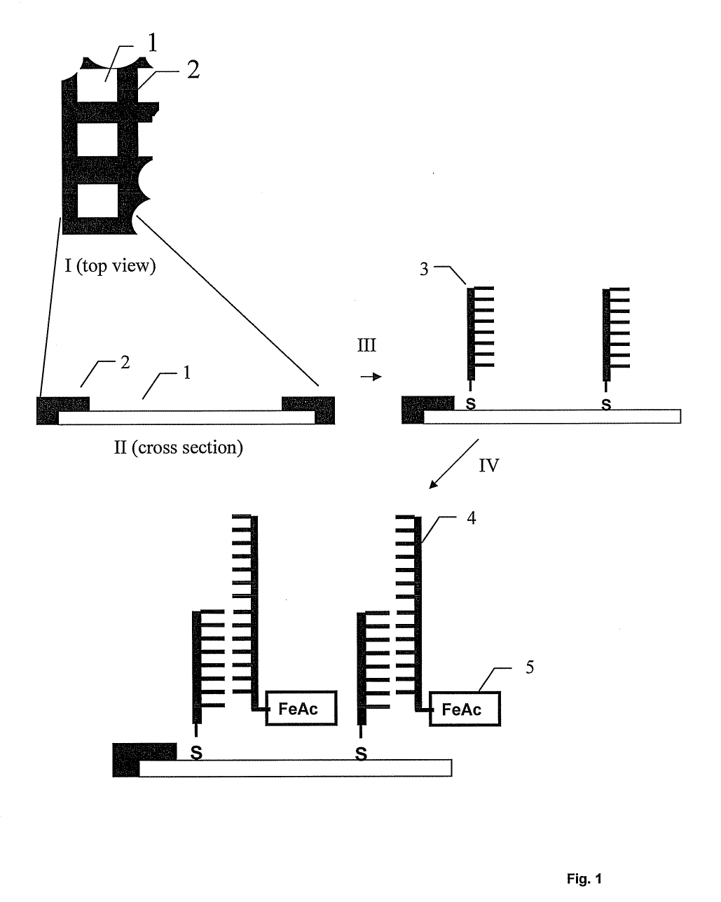

To apply the advantages of DNA chip technology to the detection of nucleic acid oligomer hybrids by the displacement assay, various modified probe nucleic acid oligomers of differing sequences are bound with the above-described immobilization techniques to a support (cf. FIG. 1, III). The arrangement of the probe nucleic acid oligomers of known sequence on defined positions on the surface, a DNA array, is intended to make the hybridization event of any target nucleic acid oligomer detectable in order to seek and sequence-specifically detect targets such as virus RNA or DNA, or also mutations in the target nucleic acid oligomer. For this purpose, the surface atoms or molecules of a defined region (a test site) on a surface are linked with DNA/RNA/PNA nucleic acid oligomers of a known but arbitrary sequence as described above. The DNA chip may also be derivatized with a single probe oligonucleotide. Nucleic acid oligomers (e.g. DNA, RNA or PNA fragments) of base length 3 to 200 or 3 to 100 or 3 to 70, preferably of length 10 to 70 or 10 to 25, are used as probe nucleic acid oligomers.

The surface thus provided, having immobilized probe oligonucleotides, is incubated with a solution of a certain quantity of signal nucleic acid oligomers, for example with redox-labeled nucleic acid oligomers (FIG. 1, IV). This leads to the formation of hybrids composed of probe nucleic acid oligomers and signal nucleic acid oligomers in the region of complementary sequences. The signal nucleic acid oligomer has a longer sequence than the probe nucleic acid oligomer and thus exhibits, beyond the hybridized region, sequence sections that—given a suitable base sequence—may serve for further hybridization, for example with a target nucleic acid oligomer (docking section). Non-bound signal nucleic acid oligomers may, if appropriate, be removed from the derivatized surface by rinsing with suitable buffer solution.

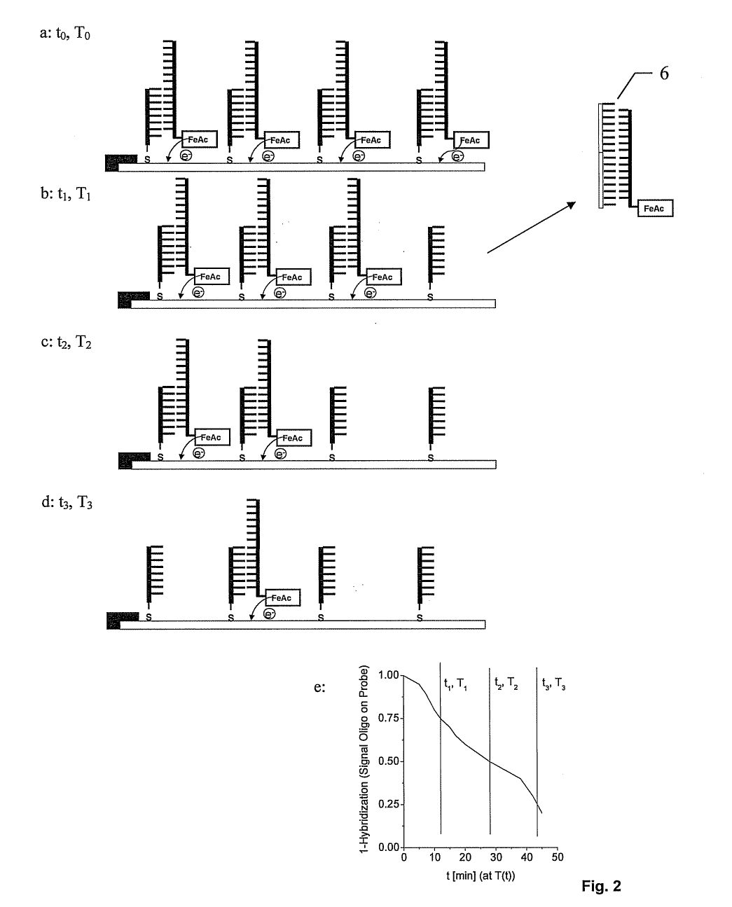

In FIG. 2, the various steps of a method according to the present invention are depicted. At a starting time t₀ (at the starting temperature T₀, prior to addition of the analytes) the signal oligonucleotides are present in a hybrid with the probe oligonucleotides immobilized on the surface (FIG. 2a). The signal oligonucleotides exhibit a longer sequence than the probe oligonucleotides and are covalently derivatized with a ferrocene (FeAc) (cf. FIG. 1). The probe oligonucleotides are immobilized to a conductive surface and hybridized with signal nucleic acid oligomer. By applying a suitable potential, ferrocene can be oxidized in an electrochemical measurement and serves as a measure for the presence of signal oligonucleotides (hybridized to probe oligonucleotides). The measurement at to and T₀ (e.g. a first cyclic voltammetric or chronocoulometric measurement) can serve as a reference measurement and determine the surface immobilized portion of signal nucleic acid oligomers.

In the next step, the test solution (as concentrated as possible) with target oligonucleotide(s) is added to the surface having immobilized probe oligonucleotides and hybridized signal nucleic acid oligomers. If the solution contains target nucleic acid oligomer strands that are complementary to the signal nucleic acid oligomers bound indirectly (by hybridization) to the surface, or complementary at least in wide regions (or wider regions than the probe oligonucleotide), a hybridization with the signal nucleic acid oligomer occurs.

Here, the target first hybridizes to the docking section of the signal oligonucleotides. The hybrid 6 composed of target and signal nucleic acid oligomer passes into the volume phase. Thereafter, the complete hybridization of target oligonucleotides and signal oligonucleotides, and displacement of the signal oligonucleotides originally hybridized to the probe oligonucleotides occurs. Thus, at a later time t₁ (at the temperature T₁, which can diverge from T₀), a part of the signal oligonucleotides of the test site is displaced, to the extent that the appropriate target was present in the analyte (FIG. 2b). This happens through hybridization of the target with the signal oligonucleotide.

At a time t₂ (at the temperature T₂, which can diverge from T₁), a further part of the signal oligonucleotides of the test site is displaced (FIG. 2c). At a later time t₃ (at the temperature T₃, which can diverge from T₂), in turn, a further part of the signal oligonucleotides of the test site is displaced (FIG. 2d).

After the hybridization of target oligonucleotides and signal oligonucleotides, in a second and possibly a series of further measurements as a function of hybridization time and temperature (e.g. a second/further cyclic voltammetric or chronocoulometric measurement), the fraction of remaining surface-immobilized signal nucleic acid oligomers is determined. For each test site, the difference resulting from the reference measurement and the second/further measurement is proportional to the number of target oligonucleotides originally present in the test solution for the respective test site. In FIG. 2e, the path of such a detection is depicted schematically.

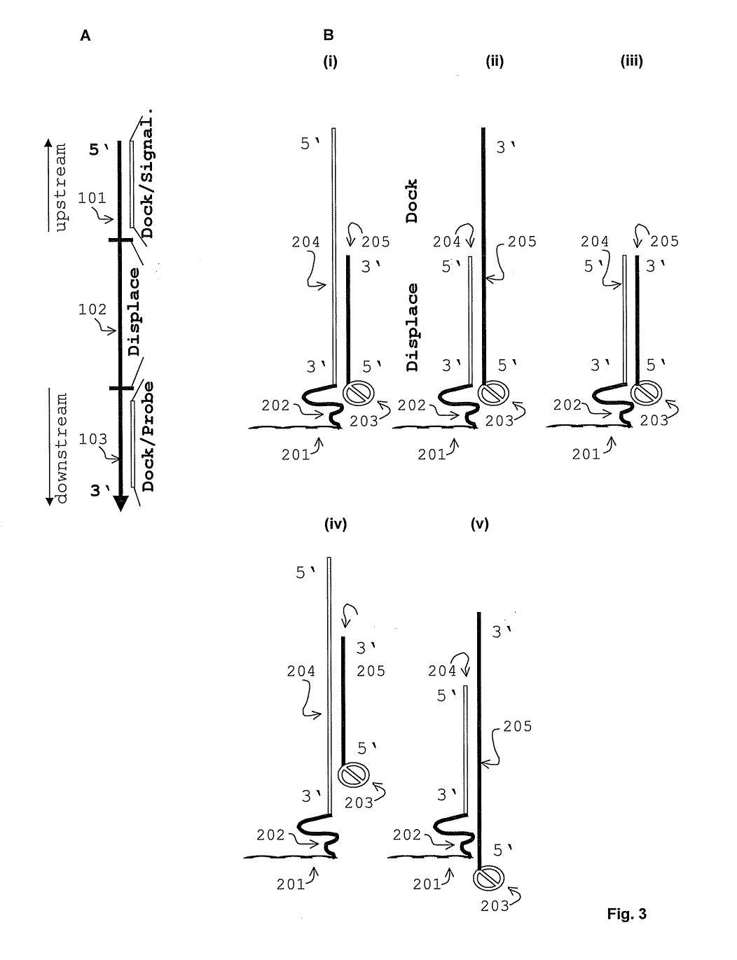

Alternatively, the reference measurement can be omitted, e.g. if the size of the reference signal is known sufficiently precisely (e.g. through preceding measurements, etc.) beforehand, or if a reference test site as depicted in FIG. 3 (iii) is present on the chip, or if multiple measurements are carried out as a function of time and/or temperature after target addition.

a) Covalent Attachment of Probe Oligonucleotides to (One) Individually Addressable Gold Electrode(s), Hybridization with Redox-Labeled Signal Nucleic Acid Oligomers, Addition of the Target Oligonucleotides and Cyclic Voltammetric Detection of the Displacement of the Signal Nucleic Acid Oligomers by the Target Nucleic Acid Oligomers:

The n-nucleotide- (nt)-long probe nucleic acid (DNA, RNA or PNA, e.g. a 20-nucleotide-long oligo) (FIG. 1, III) is provided, near one of its ends (3′- or 5′-end), directly or via a (any) spacer, with a reactive group for covalent anchoring to the surface, for example as 3′-thiol-modified probe oligonucleotide, in which the terminal thiol modification serves as a reactive group for attachment to gold electrodes. Also multiple (2 to 40) “unspecific” bases, i.e. a spacer-base sequence that is complementary or largely complementary to no sequence region of the signal nucleic acid oligomer or of the target nucleic acid oligomer, are suitable as spacers. Ideally, this “unspecific” spacer-base sequence of the probe nucleic acid oligomer is, over its entire length, not hybridizing to any sequence regions of all types of signal nucleic acid oligomers or of all types of target nucleic acid oligomers, or also to the probe nucleic acid oligomer itself (the after to avoid loops).

Further covalent anchoring options result from e.g. amino-modified probe oligonucleotide, which is used for anchoring to platinum electrodes or to glass carbon electrodes that are superficially oxidized into carboxylic acid groups. In addition, a monofunctional linker of suitable chain length with an identical reactive group may be provided. The probe nucleic acid oligomer modified in this way is,
(i) dissolved in buffer (e.g. 50-500 mM phosphate buffer, pH=7, 1 mM EDTA), brought into contact with the surface and attached there via the reactive group of the probe nucleic acid oligomer to the—if necessary, appropriately derivatized—surface or
(ii) dissolved in the presence of a monofunctional linker in buffer (e.g. 100 mM phosphate buffer, pH=7, 1 mM EDTA, 0.1-1 M NaCl), brought into contact with the surface and attached there via the reactive group of the probe nucleic acid oligomer, together with the monofunctional linker, to the—if necessary, appropriately derivatized—surface, care being taken that sufficient monofunctional linker of suitable chain length is added (about 0.1- to 10-fold or even 100-fold excess) to provide sufficient space between the individual probe oligonucleotides for hybridization with the redox-labeled signal nucleic acid oligomers or the target oligonucleotide, or
(iii) dissolved in buffer (e.g. 10-350 mM phosphate buffer, pH=7, 1 mM EDTA), brought into contact with the surface and attached there via the reactive group of the probe nucleic acid oligomer to the—if necessary, appropriately derivatized—surface. Thereafter, the surface modified in this way is brought into contact with monofunctional linker in solution (e.g. alkanethiols or w-hydroxy-alkanethiols in phosphate buffer/EtOH mixtures for thiol-modified probe oligonucleotides), the monofunctional linker attaching via its reactive group to the—if necessary, appropriately derivatized—surface.

After appropriate washing steps, the surface modified in this way is brought into contact with redox-labeled signal nucleic acid oligomers (FIG. 1, IV). As redox-labeled signal nucleic acid oligomers, for example singly or multiply ferrocene-carboxylic acid-modified 40-base-long signal oligonucleotide whose 5′-terminal sequence is complementary or largely complementary to the sequence of the probe oligonucleotide and whose complete sequence is complementary or largely complementary to the target oligonucleotide sequence may be used. Here, care is taken that significantly more labeled signal nucleic acid oligomers (at least 1.1-fold molar excess) are added than can be bound on the surface via the probe nucleic acid oligomers. After hybridization of the signal oligonucleotides to the probe oligonucleotides, excess, non-hybridized signal oligonucleotides are removed by rinsing with buffer (e.g. 350 mM phosphate buffer, pH=7, 1 mM EDTA).

Bringing the signal oligonucleotides into contact with the surface derivatized with probe oligonucleotides can occur e.g. by immersing the derivatized surface in the signal oligonucleotide solution or by spotting the derivatized surface by means of suitable methods (pipette, microdosing systems, etc.). In realizing multiple test sites for different target oligonucleotides, the immobilization of the probe oligonucleotide types (for one target each) occurs in spatially separated regions. Here, too, bringing the signal oligonucleotides into contact with the derivatized surface by immersing the derivatized surfaces (that is, all spatially separated regions with the respective probe oligonucleotides simultaneously) in the solution having the signal oligonucleotide types can occur if the sequence differences in the signal oligonucleotide types are great enough to hybridize the respective signal oligonucleotides only or at least largely only to the associated probe oligonucleotides. If this is not ensurable, the “spotting” of the individual spatially separated derivatized surfaces with the respective associated signal oligonucleotide solution is preferred. The latter method is applicable in any case.

The detection label on the signal nucleic acid oligomer is detected by a suitable method, for example by cyclic voltammetry or chronocoulometry in the case of the ferrocene-redox-labeled signal oligonucleotides (reference measurement). Thereafter, the dissolved target is added and the measurement for detecting the detection label repeated with the suitable method (multiply, as a function of time and/or temperature) (e.g. renewed cyclic voltammetric or chronocoulometric measurement in the case of the ferrocene-redox-labeled signal oligonucleotides).

The hybridization may be carried out under suitable conditions known to the person of skill in the art (any, freely choosable stringency conditions for the parameters potential/temperature/salt/chaotropic salts, etc., for hybridization). The difference between the reference measurement and measurement(s) after target addition is proportional to the number of hybrids composed of signal nucleic acid oligomers and appropriate target nucleic acid oligomers that leave the modified surface toward the volume phase.

The method may be applied for one target type, that is, a certain target oligonucleotide type having a known sequence, at one electrode, or for multiple target types, that is, differing target oligonucleotide types, at individually addressable electrodes of an electrode array that can be targeted and read out, for example via CMOS technology, in more complex arrays.

b) Indirect Attachment of Probe Oligonucleotides to Glass Fibers, Fluorophore-Labeled Signal Nucleic Acid Oligomers, Target Oligonucleotides And Fluorescence Detection of the Displacement of the Signal Nucleic Acid Oligomers by the Target Nucleic Acid Oligomers:

The n-nucleotide-long probe nucleic acid (DNA, RNA or PNA) (e.g. a 20-nucleotide-long oligo) is provided near one of its ends (3′- or 5′-end), directly or via a (any) spacer with a reactive group for covalent anchoring to the surface, e.g. a carboxy-modified probe oligonucleotide for attachment to amino-modified silanized glass or silicon (e.g. to (3-aminopropyl)-triethoxysilane-modified glass or silicon). Further covalent anchoring options result from e.g. amino-modified probe oligonucleotide, which is used for anchoring to dextran polymers that are derivatized with carboxylic acid and immobilized on glass or silicon, the thickness of the layer composed of dextran polymer with attached probe oligonucleotides in this embodiment being able to be varied via the dextran polymer composition, dextran anchor groups at the glass surface, anchor groups of the dextran for immobilization on the glass, incubation duration on the glass, etc., using methods known to the person of skill in the art. In a preferred embodiment, the thickness of the dextran/probe oligonucleotide layer is chosen such that it approximately corresponds to the penetration depth of the evanescent field of the light for exciting the fluorophores (approx. 50 nm to approx. 500 nm, depending on whether a metallization layer is located on the glass to increase the penetration depth of the evanescent field).

The probe nucleic acid modified in this way is dissolved in buffer (e.g. 50-500 mM phosphate buffer, pH=7, 1 mM EDTA) in the presence of EDC and sNHS (each approximately 40-fold molar excess in relation to the probe oligonucleotide), brought into contact with the modified glass (or silicon) surface and attached there to the surface via the reactive group of the probe nucleic acid oligomer (if necessary, nonfunctional surface binding sites are treated beforehand with a suitable blocking reagent).

After suitable washing steps, the surface modified in this way is brought into contact with fluorophore-labeled signal nucleic acid oligomers, similarly as described under embodiment a). As fluorophore-labeled signal nucleic acid oligomers, for example fluorescein-modified nucleic acid oligomers (e.g. of base length 40) may be used whose sequence at the 5′-terminal side is complementary or largely complementary to the sequence of the probe oligonucleotide and whose complete sequence is complementary or largely complementary to the target oligonucleotide sequence. Here, care is taken that significantly more labeled signal nucleic acid oligomers (at least 1.1-fold molar excess) are added than can be bound on the surface via the probe nucleic acid oligomers. After hybridization of the signal oligonucleotides to the probe oligonucleotides, excess, non-hybridized signal oligonucleotides are removed by rinsing with buffer (e.g. 350 mM phosphate buffer, pH=7, 1 mM EDTA).

The detection label on the signal oligonucleotide is detected by a suitable method, e.g. by total internal reflection fluorescence (TIRF) in the case of the fluorophore-labeled signal nucleic acid oligomers (reference measurement). Thereafter, the test solution is added and potential hybridization events are made possible under suitable conditions known to the person of skill in the art (any, freely choosable stringency conditions for the parameters potential/temperature/salt/chaotropic salts, etc., for hybridization). Following this, the measurement for detecting the detection label is repeated with the suitable method (multiply, as a function of time and/or temperature) (e.g. renewed TIRF measurement in the case of the fluorescein-labeled signal nucleic acid oligomers). The difference between the reference measurement and the measurements after target addition is proportional to the number of hybridization events between signal nucleic acid oligomer and appropriate target nucleic acid oligomer in the test solution that leave the surface as a hybrid. In detection by determining the TIRF, a decrease in the fluorescence signal is to be expected.

The method may be applied for one target type, that is, a certain target oligonucleotide type having a known sequence, for example on a glass fiber, or for multiple target types, that is, differing target oligonucleotide types, for example on individually addressable glass fibers of a glass fiber bundle.

### c) Embodiments of the Displacement Assay and Possible Reference Test Sites (FIG. 3):

The single strand that is to be captured via the assay is referred to as the target nucleic acid oligomer (sequence 5′ to 3′). It comprises a sequence section that preferably serves to identify/attach to the signal oligo and to discriminate from other targets (“displace” region). Directly or indirectly adjoining sequence regions “upstream or downstream” therefrom may be drawn on to make hybridization to the docking section of the signal nucleic acid oligomer possible.

In FIG. 3B, two principle options (i) and (ii) are depicted for realizing, with a non-hybridized docking region for the target, the hybrid composed of probe and annealed signal nucleic acid oligomer immobilized on the surface. For comparison, FIG. 3B (i) shows the case in which the docking region is located on the probe nucleic acid oligomer. The 5′-end of the probe nucleic acid oligomer clearly projects beyond the 3′-end of the signal nucleic acid oligomer and provides the docking region in the projecting section. In FIG. 3B (ii) is depicted that the docking region is located according to the present invention on the signal nucleic acid oligomer. The 3′-end of the signal nucleic acid oligomer clearly projects beyond the 5′-end of the probe nucleic acid oligomer and provides the docking region in the projecting section.

In addition to the hybridization regions between the probe and signal oligo, depicted in FIG. 3B (i) and (ii), also any other hybrid structure may be drawn on. For example, starting from the structure depicted in (i), the signal oligo—given an appropriately modified sequence—may be shifted toward 3′ or 5′ of the probe until, in the extreme case, it sits at the 5′-terminal end (or extends beyond it). Such a situation is depicted in FIG. 3B (iv). In this case, on the probe nucleic acid oligomer are located two docking regions, one beginning at the 5′-end of the probe nucleic acid oligomer toward the 3′-end of the signal oligo, and a second beginning at the 3′-end of the probe nucleic acid oligomer toward the 5′-end of the signal oligo. FIG. 3B (v) shows the analogous case for two docking sections on the signal nucleic acid oligomer.

Thus, in (i), the docking section (non-hybridized region in the adduct composed of probe and signal nucleic acid oligomer) lies on the probe nucleic acid oligomer. After probe addition—given the appropriate base sequence of this section—a sequence region 103 of the target hybridizes to this docking section. The same applies analogously for (ii), such that here, the 3′-end of the signal oligo hybridizes e.g. with the 5′-end of the probe or even hybridizes further downstream from the probe sequence. In case (ii), the docking section (non-hybridized region in the adduct composed of probe and signal nucleic acid oligomer) lies on the signal nucleic acid oligomer. After probe addition—given the appropriate base sequence of this section—a sequence region 101 of the target hybridizes to this docking section.

In addition to the actual test site according to one of the variations in (i) and (ii), a reference test site (iii) may be realized (e.g. as an on-chip reference): for this, for any sequence of the hybrid composed of probe and signal nucleic acid oligomers in cases (i) and (ii), the so-called “inverse” sequences of the hybrid are used to construct a probe with associated signal oligonucleotide of identical base length, but that exhibits no docking site and an inverse base sequence. Theoretically, the melting curve of the inverse hybrid exhibits approximately the same path (signal vs. temperature) as the hybridized portion in (i) or (ii). The base sequence, which is normally given in 5′ to 3′, is retained as the inverse sequence, but in the inverse direction (3′ to 5′). For example, the sequence 3′-GTTCAAAG-5′ (and thus 5′-GAAACTTG-3′) is inverse to the sequence 5′-GTTCAAAG-3′.

### Example 1

### Preparing the N-Hydroxysuccinimide Active Ester of the Redox (or Fluorophore) Label

1 mmol of the respective carboxylic acid derivative of a fluorophore (e.g. fluorescein) or of a redox-active substance (e.g. ferrocene) and 1.1 mmol N-hydroxysuccinimide are dissolved in 15 ml anhydrous dioxane. 1.1 mmol carbodiimide (dissolved in 3 ml anhydrous dioxane) are cooled with ice and added dropwise to the carboxylic acid derivative. The reaction mixture is stirred for 16 h at RT, the resultant precipitate filtered off and the solvent drawn off. The residue is purified by silica gel chromatography (Merck silica gel 60, eluent: dichloromethane/ethyl acetate/heptane mixtures).

### Example 2

### Preparing the Amino-Modified Oligonucleotides for Coupling the Active Ester Label of Ex. 1, or Thiol-Modified Oligonucleotides for Anchoring on Gold as Probe Nucleic Acid Oligomers

The synthesis of the oligonucleotides occurs in an automatic oligonucleotide synthesizer (Expedite 8909; ABI 384 DNA/RNA synthesizer) according to the synthesis protocols recommended by the manufacturer for a 1.0 μmol synthesis. As standard, the synthesis of the signal nucleic acid oligomers takes place on A-CPG as the support material. Modifications at the 5′-position of the oligonucleotides occur with a coupling step prolonged to 5 minutes. The amino modifier C2 dT (Glen Research 10-1037) is built into the sequences with the respective standard protocol.

The constitution of 3′-dithiol-modified probe oligonucleotides occurs on a 5-hydroxy-1,2-dithiane-4-O-dimethoxytrityl-modified CPG support, and further dithiol-modifications occur by means of 1,2-dithiane-4-O-dimethoxytrityl-5-[(2-cyanoethyl)-N,N-diisopropyl)]-phosphoramidite (DTPA, Glen Research 10-1937) analogously to standard protocols, the oxidation steps being carried out with a 0.02 M iodine solution to avoid oxidative cleavage of the disulfide bridge(s). The coupling efficiencies are determined online during the synthesis, photometrically or conductometrically, via the DMT cation concentration.

The oligonucleotides are deprotected with concentrated ammonia (30%) at 37° C. over a period of 16 h. The purification of the oligonucleotides occurs by means of RP-HPL chromatography according to standard protocols (eluent: 0.1 M triethylammonium acetate buffer, acetonitrile), the characterization by means of MALDI-TOF MS.

### Example 3

### Converting the Amino-Modified Oligonucleotides (Ex. 2) with the N-Hydroxy Active Esters (Ex. 1)

The amino-modified oligonucleotides are dissolved in 0.1 M borate buffer (pH 8.5) and converted with the N-hydroxysuccinimide active esters dissolved in DMSO according to the protocol from Molecular Probes (Labeling Amine-Modified Oligonucleotides). The purification of the oligonucleotides occurs by means of RP—HPL chromatography according to standard protocols (eluent: 0.1 M triethylammonium acetate buffer, acetonitrile), the characterization by means of MALDI-TOF MS.

### Example 4

### Manufacturing the Probe and Signal Oligonucleotides for an HPV 6 Assay

For the detection of HPV_{—}6 in the form of a displacement assay according to an embodiment of the present invention, the following sequences were synthesized in accordance with ex. 1 to 3:

Probe HPV_6:
5′-cgta T ctacatcttccac T tacaccaa CATATTTATT-(S-
S)₃-3′
Signal oligo HPV_6:
5′-(Fc)₄-ttggtgtatgtggaagatgtagttacg_gatgtacataatg
tcatgttggtactgcg-3′
Probe HPV_6_Ref:
5′-ttggtgta A gtggaagatgtag A tacg TCAATTTTTTT-(S-
S)₃-3′
Signal oligo*HPV_6_Ref
5′-(Fc)₄-cgtaactacatcttccacatacaccaa

Here, in the probes, the capital letters stand for the “unspecific” spacer-base sequence. Bases in capital letters and underscores are not complementary to the signal oligonucleotide. (S-S)₃ stands for three DTPA units (see ex. 2) with a total of six thio functions for immobilizing the probe on the gold electrode. In the signal oligonucleotides, (Fc)₄ stands for four ferrocene labels covalently attached to the signal olignucleotide. For the signal oligo HPV-6, the region for hybridization to the probe and the docking region are separated by an underscore (sequence section for the probe hybridization_sequence section of the docking region).

### Example 5

### Manufacturing the Oligonucleotide-Electrode “HPV_{—}6” and “HPV_{—}6_Ref”

A gold microelectrode within an electrode array forms the support material for the covalent attachment of the double-strand oligonucleotides. The gold surface is freed from surface impurities immediately before incubation with the probes (probe HPV_{—}6 or probe HPV_{—}6_Ref, cf. ex. 2 and 4) in 0.5 M H₂SO₄ as the electrolyte, through cyclic voltammetric measurements in the range 0 to 1.35 V vs. Ag/AgCl. After this process, generally referred to as electropolishing, the electrode array is rinsed with water and thereafter with ethanol, and residual ethanol traces are removed in an Ar stream.

For incubation, probe HPV_{—}6 or probe HPV_{—}6_Ref as a 5×10⁻⁵ molar solution is dissolved in 500 mM Na-phosphate buffer, pH 7, and applied with microdosing systems to one test site each of the electrode array, such that the gold surface of a test site is completely wetted, and incubated for 2 h. The contact or non-contact printing used for this is described in detail in WO 2004/082814 A2, to which reference is made in this context. During this reaction time, the disulfides of the DTPA units (cf. ex. 2 and 4) are anchored to the gold surface via chemisorption.

Thereafter, the surface modified in this way is rinsed with buffer (500 mM Na-phosphate, pH=7) and subsequently incubated with 6-hydroxy-hexanethiol in 250 mM phosphate buffer, pH=7, mit 1% ethanol for 6 h to passivate free gold (attachment of the alkanethiols to gold via chemisorption).

After alkanethiol incubation has occurred, the electrode array is rinsed again (with 250 mM Na-phosphate buffer, pH=7, thereafter with ethanol), blown dry in an Ar stream and thereafter incubated with a solution of the signal oligonucleotides (signal-oligo HPV_G and signal-oligo HPV_{—}6_Ref, cf. ex. 2 and 4) (50 nM in Na-phosphate buffer, pH=7) for approx. 20 min., rinsed again (350 mM Na-phosphate buffer, pH=7) and dried in an Ar stream.

### Example 6

### Displacement Assay for HPV-6 with Temperature Gradient

The chip obtained in ex. 5 is measured in electrolyte (250 mM Na₂SO₄) cyclic voltammetrically (Potentiostat Autolab PGSTAT 12 from Metrohm, potential range 0 to 0.5 V vs. Ag/AgCl, scan rate 500 mV/s, room temperature, here 26° C.). A current-voltage curve is obtained in this measurement, the peak current of the cyclic voltammogram obtained being proportional to the number of (oxidizable) ferrocenes and thus proportional to the number of signal oligonucleotides and thus proportional to the number of hybrids composed of signal and probe oligonucleotide. The measurement obtained in this way serves to determine the reference value (reference measurement).

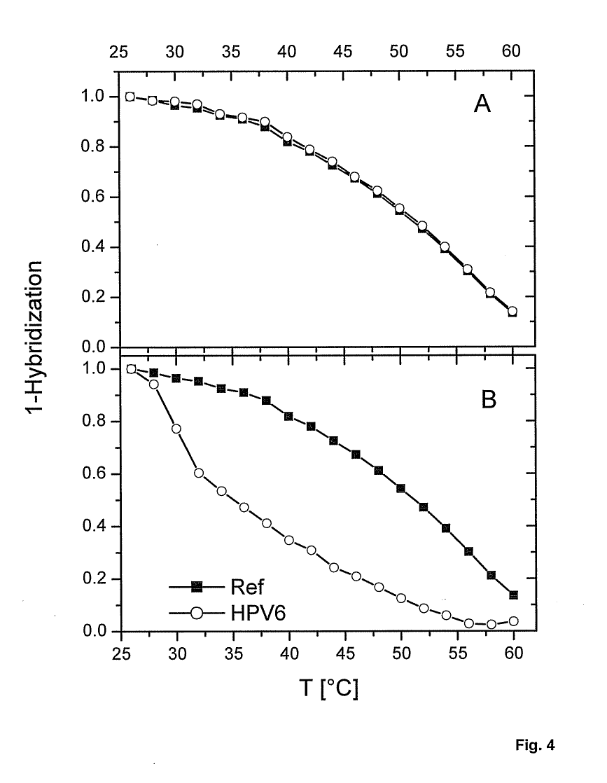

Thereafter, the electrolyte, and thus the electrodes, are heated up at 2° C./min and the measurements are repeated multiple times under otherwise identical conditions. FIG. 4A depicts the measurement result of this procedure, normalized to the reference value. The curve path substantially corresponds to a melting curve of the hybrids composed of probe and signal oligonucleotide.

The same measurement procedure is subsequently repeated, with, however, after the reference measurement, an HPV_{—}6 target nucleic acid oligomer being added to the electrolyte (asymmetric PCR product in water, 130 bases long with a region that is complementary to the signal oligonucleotide sequence). In the measurement in FIG. 4 B, the target was present in a concentration of approx. 10 nM.

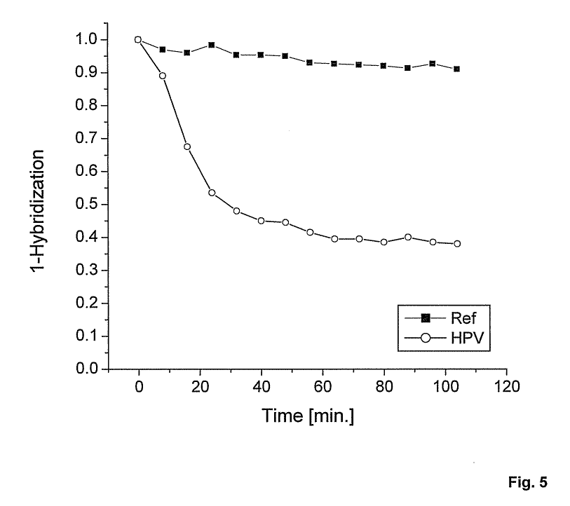

Instead of with a temperature gradient, the measurements can be carried out with and without target also as follows. First, a reference measurement is carried out, and thereafter, the hybridization between target and signal oligonucleotide is tracked at a suitable temperature through repeated measurement. In the case of the measurements depicted in FIG. 5, the reference measurement occurred at room temperature, the further measurements as a function of time at 38° C. Otherwise, the conditions corresponded to the conditions described above in connection with the measurements depicted in FIG. 4.

1. A method for detecting nucleic acid oligomer hybridization events, comprising the steps
a) modifying a surface by attaching to the surface at least one type of probe nucleic acid oligomer,
b) providing at least one type of signal nucleic acid oligomer, the signal nucleic acid oligomers being modified with at least one detection label and the signal nucleic acid oligomers having a section that is complementary or substantially complementary to the probe nucleic acid oligomers,
c) providing a sample having target nucleic acid oligomers,
d) bringing a defined quantity of the signal nucleic acid oligomers into contact with the modified surface,
e) bringing the sample and the target nucleic acid oligomers contained therein into contact with the modified surface,
f) detecting the signal nucleic acid oligomers,
g) comparing the values obtained in step 0 with reference values,
wherein the signal nucleic acid oligomers have a larger number of bases than do the probe nucleic acid oligomers, and the signal nucleic acid oligomers exhibit at least one docking section, the docking section exhibiting no structure that is complementary or largely complementary to a section of the probe nucleic acid oligomers, and the target nucleic acid oligomers having a section that is complementary or largely complementary to the docking section.

2. The method according to claim 1, wherein the number of bases of probe nucleic acid oligomers and signal nucleic acid oligomers differs by 6 to 80.

3. The method according to claim 1, wherein the number of bases of probe nucleic acid oligomers and signal nucleic acid oligomers differs by 9 to 60.

4. The method according to claim 1, wherein the number of bases of probe nucleic acid oligomers and signal nucleic acid oligomers differs by 10 to 40.

5. The method according to claim 1, further comprising, after step d) and before step e), the step of d₁) detecting the signal nucleic acid oligomers to determine reference values, and wherein step g) comprises comparing the values obtained in step f) with the reference values obtained in step d₁).

6. The method according to claim 1, wherein the signal nucleic acid oligomers comprise 10 to 200 nucleic acids.

7. The method according to claim 1, wherein the signal nucleic acid oligomers comprise 20 to 100 nucleic acids.

8. The method according to claim 1, wherein the signal nucleic acid oligomers comprise 25 to 70 nucleic acids.

9. The method according to claim 1, wherein the signal nucleic acid oligomers and the probe nucleic acid oligomers exhibit at least one non-complementary base pair in one or more regions of their sequence that hybridize with each other.

10. The method according to claim 1, further comprising repeating the step of detecting the signal nucleic acid oligomers multiple times.

11. The method according to claim 10, wherein a first instance of detecting the signal nucleic acid oligomers occurs in a buffer solution before the sample is brought into contact with the modified surface.

12. The method according to claim 10, wherein a first instance of detecting the signal nucleic acid oligomers, after said step of bringing the sample and the target nucleic acid oligomers into contact with the modified surface, is carried out immediately after the sample is brought into contact with the modified surface and, thereafter, is repeated multiple times over a period of at least 40 min.

13. The method according to claim 12, further comprising repeating the step of detecting the signal nucleic acid oligomers multiple times over a period of at least 20 minutes.

14. The method according to claim 12, further comprising repeating the step of detecting the signal nucleic acid oligomers multiple times over a period of at least 10 minutes.

15. The method according to claim 12, further comprising repeating the step of detecting the signal nucleic acid oligomers multiple times over a period of at least 5 minutes

16. The method according to claim 10, comprising, during the step of repeating the detecting of the signal nucleic acid oligomers multiple times, the step of raising the temperature in the region of the modified surface, starting at room temperature.

17. The method according to claim 16, wherein the step of changing the temperature in the region of the modified surface comprises raising the temperature above room temperature at a rate ranging between 1° C. per minute to 10° C. per minute.

18. The method according to claim 16, wherein the step of changing the temperature in the region of the modified surface comprises raising the temperature above room temperature at a rate of about 2° C. per minute.

19. A method for detecting nucleic acid oligomer hybridization events, comprising the steps
a) providing a modified surface, the modification consisting in the attachment of at least one type of probe nucleic acid oligomer, wherein at least one type of signal nucleic acid oligomer modified with at least one detection label being present hybridized to the probe nucleic acid oligomers
b) providing a sample having target nucleic acid oligomers,
c) bringing the sample and the target nucleic acid oligomers contained therein into contact with the modified surface,
d) detecting the signal nucleic acid oligomers,
e) comparing the values obtained in step f with reference values,
wherein the signal nucleic acid oligomers have a larger number of bases than do the probe nucleic acid oligomers, and the signal nucleic acid oligomers exhibit at least one docking section, the docking section exhibiting no structure that is complementary or largely complementary to a section of the probe nucleic acid oligomers, and the target nucleic acid oligomers having a section that is complementary or largely complementary to the docking section.

20. A modified surface comprising
at least one type of probe nucleic acid oligomer attached to the surface,
at least one type of signal nucleic acid oligomer modified with at least one detection label, wherein the signal nucleic acid oligomer is hybridized to the probe nucleic acid oligomer,
wherein the signal nucleic acid oligomers have a larger number of bases than the probe nucleic acid oligomers and the signal nucleic acid oligomers exhibit at least one docking section, the docking section exhibiting no structure that is complementary or largely complementary to a section of the probe nucleic acid oligomers.

21. The modified surface according to claim 21, wherein said signal nucleic acid oligomers and probe nucleic acid oligomers exhibit at least one non-complementary base pair in one or more regions of their sequences that hybridize with each other.

22. A kit for carrying out a method for detecting nucleic acid oligomer hybridization events, comprising a modified surface, the modification consisting in attaching at least one type of probe nucleic acid oligomer, and an effective quantity of signal nucleic acid oligomers, wherein the signal nucleic acid oligomers have a larger number of bases than do the probe nucleic acid oligomers, and the signal nucleic acid oligomers exhibit at least one docking section, the docking section exhibiting no structure that is complementary or largely complementary to a section of the probe nucleic acid oligomers, and the target nucleic acid oligomers having a section that is complementary or largely complementary to the docking section.
